(19) United States
(12) Patent Application Publication
(10) Pub. No.: US 2018/0200312 A1
(43) Pub. Date: Jul. 19, 2018

(54) Methods and Compositions for Enhancing Memory and/or Reducing Fear and/or Pain of a Host by Administering a Probiotic

(71) Applicants: The Regents of the University of California, Oakland, CA (US); Battelle Memorial Institute, Richland, WA (US)

(72) Inventors: Antoine M. Snijders, Antioch, CA (US); Jian-Hua Mao, Moraga, CA (US); Sasha A. Langley, Oakland, CA (US); Thomas O. Metz, Kennewick, WA (US); Janet K. Jansson, Pasco, WA (US)

(73) Assignees: The Regents of the University of California, Oakland, CA (US); Battelle Memorial Institute, Richland, WA (US)

(21) Appl. No.: 15/873,823

(22) Filed: Jan. 17, 2018

Related U.S. Application Data
(60) Provisional application No. 62/447,417, filed on Jan. 17, 2017.

(51) Int. Cl. A61K 35/747; A61K 35/741; A61K 9/00

(52) U.S. Cl. CPC A61K 35/747; A61K 9/0053; A61K 35/741

(57) ABSTRACT

The present invention provides for a probiotic composition designed to enhance memory and/or reduce fear and/or pain of a host, the probiotic composition comprising viable or live cells of one or more of the family/order/strain selected from a group consisting of RF39, Lactobacillaceae, Bacteroidaceae, Lachnospiraceae, Anaeroplasmataceae, Ruminococcaceae, Clostridiales, Clostridiaceae, Rikenellaceae, Erysipelotrichaceae, Peptococcaceae, Turicibacteraceae, Deferribacteraceae, Cytophagaceae, Chitinophagaceae, Coriobacteriaceae, Bifidobacteriaceae, and S24.7. The present invention also provides for a method of enhancing memory and/or reducing fear and/or pain of a host, the method comprising: administering the probiotic composition to a host.

### RELATED PATENT APPLICATIONS

The application claims priority to U.S. Provisional Patent Application Ser. No. 62/447,417, filed Jan. 17, 2017; which is incorporated herein by reference.

### REFERENCE TO SEQUENCE LISTING, TABLE, OR COMPUTER PROGRAM APPENDIX

The application incorporates by reference the attached slides and supplementary tables appendices of U.S. Provisional Patent Application Ser. No. 62/447,417, filed Jan. 17, 2017.

### STATEMENT OF GOVERNMENTAL SUPPORT

This invention was made during work supported by U.S. Department of Energy and by Laboratory Directed Research and Development program Microbes to Biomes (LBNL) Initiative under Contract No. DE-AC02-05CH11231, and by Laboratory Directed Research and Development Microbiomes in Transition (PNNL) program under DOE Contract No. DE-AC05-76RLO 1830, and by Office of Naval Research under ONR contract N0001415IP00021. The government has certain rights in this invention.

### BACKGROUND OF THE INVENTION

### Field of the Invention

The present invention relates to methods of use and compositions of probiotics.

### BRIEF SUMMARY OF THE INVENTION

The present invention provides for a probiotic composition designed to enhance memory and/or reduce fear and/or pain of a host, the probiotic composition comprising viable or live cells of one or more of the family/order/strain selected from a group consisting of RF39, Lactobacillaceae, Bacteroidaceae, Lachnospiraceae, Anaeroplasmataceae, Ruminococcaceae, Clostridiales, Clostridiaceae, Rikenellaceae, Erysipelotrichaceae, Peptococcaceae, Turicibacteraceae, Deferribacteraceae, Cytophagaceae, Chitinophagaceae, Coriobacteriaceae, Bifidobacteriaceae, and S24.7.

In some embodiments, the probiotic composition comprises viable or live cells of two or more of the family/order/strain selected from the group. In some embodiments, the probiotic composition comprises viable or live cells of three or more of the family/order/strain selected from the group. In some embodiments, the probiotic composition comprises viable or live cells of four or more of the family/order/strain selected from the group. In some embodiments, the probiotic composition comprises viable or live cells of five or more of the family/order/strain selected from the group. In some embodiments, the probiotic composition comprises viable or live cells of six or more of the family/order/strain selected from the group. In some embodiments, the probiotic composition comprises viable or live cells of seven or more of the family/order/strain selected from the group. In some embodiments, the probiotic composition comprises viable or live cells of eight or more of the family/order/strain selected from the group. In some embodiments, the probiotic composition comprises viable or live cells of nine or more of the family/order/strain selected from the group.

In some embodiments, the probiotic composition comprising viable or live cells of one or more of the family/order/strain RF39, Lactobacillaceae, Bacteroidaceae, Lachnospiraceae, Anaeroplasmataceae, Ruminococcaceae, Clostridiales, Clostridiaceae, or Rikenellaceae.

In some embodiments, the probiotic composition comprises viable or live cells of strain RF39 and/or family Lactobacillaceae. In some embodiments, the probiotic composition further comprises viable or live cells of the family Bacteroidaceae. In some embodiments, the probiotic composition further comprises viable or live cells of the family Lachnospiraceae. In some embodiments, the probiotic composition further comprises viable or live cells of the family Anaeroplasmataceae. In some embodiments, the probiotic composition further comprises viable or live cells of the family Ruminococcaceae. In some embodiments, the probiotic composition further comprises viable or live cells of the order Clostridiales. In some embodiments, the probiotic composition further comprises viable or live cells of the family Clostridiaceae. In some embodiments, the probiotic composition further comprises viable or live cells of the family Rikenellaceae.

The present invention provides for a method of enhancing memory and/or reducing fear and/or pain of a host, the method comprising: administering a probiotic composition of the present invention to a host, such that memory is enhanced, and/or fear and/or pain is reduced, for the host.

In some embodiments, the host is a mammal. In some embodiments, the host is a mouse. In some embodiments, the host is a primate. In some embodiments, the host is a human.

In some embodiments, the administering is oral administration.

Although the gut microbiome plays important roles in host physiology, health and disease¹, we lack understanding of the complex interplay between host genetics and early life environment on the microbial and metabolic composition of the gut. We used the genetically diverse Collaborative Cross mouse system² to discover that early life history impacts the microbiome composition, whereas dietary changes have only a moderate effect. By contrast, the gut metabolome was shaped mostly by diet, with specific non-dietary metabolites explained by microbial metabolism. Quantitative trait analysis identified mouse genetic trait loci (QTL) that impact the abundances of specific microbes. Human orthologues of genes in the mouse QTL are implicated in gastrointestinal cancer. Additionally, genes located in mouse QTL for Lactobacillales abundance are implicated in arthritis, rheumatic disease and diabetes. Furthermore, Lactobacillales abundance was predictive of higher host T-helper cell counts, suggesting an important link between Lactobacillales and host adaptive immunity.

### BRIEF DESCRIPTION OF THE DRAWINGS

FIG. 1A, Schematic of the study design.

FIG. 1B, Normalized relative abundance of the most common genera in the two built environments (BEs), colored by order and separated at family level.

FIG. 1C, Differentially abundant faecal OTUs between animal facility BEs (BE1 versus BE2 at UNC; α=0.01).

FIG. 1D, Heatmap of differential abundance of taxa between BE1 and BE2 across 30 mouse strains.

FIG. 1E, The distinct microbiome established at birth (BE1 and BE2) is sustained after transfer to a novel environment (BE3) and passed to the second generation born in BE3. Samples are color coded by BE at UNC (red, BE1; blue, BE2) in this multidimensional scaling ordination of Bray-Curtis distances between normalized samples. The ADONIS test was used to assess the statistical significance of clustering based on BE (P<0.001, R²=0.09617 blocking by time point); the built environment BE effects were also significant when tested separately at each time point (all P<0.02).

FIG. 2A, Genomic architecture of QTL for gut microbiome composition. The outer layer shows chromosome location (each chromosome is uniquely colored and labelled; major tick marks within each chromosome arm correspond to 25 Mb). The second layer (grey) shows the number of OTUs at each SNP that reach QTL significance (Mann-Whitney U test, P≤1×10⁻⁶). The third layer (red) shows the genomic intervals based on QTL significance for ≥10 OTUs. The fourth layer (green) shows the number of OTUs at each SNP that reach QTL significance for Clostridiales (family (f.) unknown), with the red line indicating 10 OTUs. The 5th-11th layers represent genomic intervals based on QTL significance for Clostridiales f. unknown, Clostridiaceae and Lachnospiraceae, Deferribacterales f. Deferribacteraceae, Bifidobacteriales f. Bifidobacteriaceae, Lactobacillales f. Lactobacillaceae and Anaeroplasmatales f. Anaeroplasmataceae, respectively.

FIG. 2B, Manhattan plot of the GWAS analysis. OTUs are merged at the family level for Lactobacillaceae with the x axis showing genomic location and the y axis showing the association level. The −log₁₀(P value) is shown for 20,199 SNPs ordered by genomic position. The horizontal black line indicates the QTL significance threshold at −log₁₀(P value)=6. Candidate genes located in representative QTL are listed above the plot.

FIG. 2C, SNP-specific association with Lactobacillaceae abundance for examples on chromosomes 1 (191,968,724 bp), 10 (66,380,845 bp), 16 (22,739,388 bp) and 17 (27,818,729 bp) (whiskers represent 5th and 95th percentiles).

FIG. 3A, Random forest analysis to assess the association between microbial abundance at the family level and mouse peripheral blood T (CD3³⁰/CD45R⁻), T-helper (CD3⁺/CD45R⁻/CD4⁺/CD8⁻) and T-suppressor (CD3⁺/CD45R⁻/CD4⁻/CD8⁺) cell counts. Significant associations are indicated in red (P<0.05).

FIG. 3B, Human homologues of candidate genes within joint QTL intervals defined in FIG. 2A (third layer) are significantly enriched for genes implicated in gastrointestinal (GI) tract cancer, lipid metabolism and immune system functions (ingenuity pathway analysis, IPA).

FIG. 3C, Human homologues of candidate genes in QTL for Deferribacteraceae, Clostridiales f. unknown, Clostridiacea, Anaeroplasmataceae, Bifidobacteriaceae, Lachnospiraceae and Lactobacillaceae are significantly enriched for GI tract cancer (blue bars indicate −log₁₀(P value)>6), while remaining families do not show significant enrichment. Candidate genes in QTL for Lactobacillaceae are also significantly enriched for arthritis, diabetes and chronic inflammatory disorder (red bars).

FIG. 3D, Human homologues of candidate genes within joint QTL intervals defined in FIG. 2A (third layer, 1,215 unique human homologues) showed significant overlap with GWASs for Crohn's disease (41 genes), coeliac disease (7 genes), ulcerative colitis (12 genes), body mass index (20 genes), type 2 diabetes (14 genes) and metabolite levels in blood serum or cerebral spinal fluid (19 genes). Human gut-related diseases are indicated in orange. Horizontal red line indicates significance threshold for overlap at −log₁₀(P value)>1.3.

FIG. 4A, Top: diet is the main contributor to metabolite profiles. Bottom: GC-MS chromatograms of diet 1 and diet 2. PCoA of metabolite profiles were measured in faecal samples of 24 CC strains maintained on two different diets (P<0.001, R²=0.16597, ADONIS).

FIG. 4B, Relative abundance of select metabolites in faecal samples from individual mice fed on different diets. Error bars indicate mean±s.e.m.

FIG. 4C, Diet is a primary contributor to metabolite profiles and correlates strongly with principal coordinate 1. Metabolite profiles were measured in faecal samples of four CC strains (males and females were analyzed separately for each strain) maintained for one week on standard chow (Labdiet Picolab 5053; diet 1) or one week on autoclaved chow (Labdiet Prolab 3500; diet 2), followed by one week on standard chow (P<0.001, R²=0.14237 between two diets, ADONIS).

FIG. 4D, Metabolic modelling-based taxonomic contributors to metabolite variation for mice on the autoclaved Labdiet Prolab 3500 chow (BE1 and BE2). Individual OTUs shown (circles; colored at the family level) are those whose metabolic capacity and variation across samples are consistent with the metabolic potential of the entire community and with measured variation in the linked metabolites (squares). Green and orange clouds behind OTU sub-networks indicate Clostridiales and Bacteroidales enrichment. Edge color indicates whether a given OTU potentially impacts a certain metabolite variation via synthesis (blue edges), degradation (green edges) or both (purple edges).

FIG. 5A, Collaborative Cross (CC) mice strains and the corresponding results from the testing of latency to enter. The orange bars indicate the latency to enter (in seconds) for each strain at day 0 when the mice are first trained. The dark blue bars indicate the latency to enter (in seconds) for each strain 72 hours after training.

FIG. 5B, Random forest analysis to assess the association between microbial abundance at the family level and latency to enter. Significant associations are indicated in yellow.

### DETAILED DESCRIPTION OF THE INVENTION

### Introduction

Atypical sleep schedules and other types of stress in certain environments, where circadian rhythm is altered, can impact host physiology. Physiological impacts can include gastrointestinal illness, coronary artery diseases, depression and reduced memory, memory function, alertness and cognitive performance, and increased risk for diabetes and obesity.

The following studies and findings shows that stress experienced by individuals due to disrupted circadian rhythm is intimately associated with gut microbiome. By gaining better understanding of gut microbiome function and signaling between gut and brain, we will be able to devise strategies to mitigate deleterious stress symptoms. Development of novel probiotic compositions and methods of using such compositions that influence host phenotypes (for example memory) can have broad applications.

Snijders A M, Langley S A, Kim Y M, Brislawn C J, Noecker C, Zink E M, Fansler S J, Casey C P, Miller D R, Huang H, Karpen G H, Celniker S E, Brown J B, Borenstein E, Jans son J K, Metz T O, Mao J H. Nature Microbiology, 2016 Nov. 28; 2:16221, describe the present methods and compositions and is hereby incorporated by reference in its entirety including the supplemental information found at found at website for: nature.com/articles/nmicrobio12016221#supplementary-information, incorporated by reference., for all purposes.

All references to any Supplementary Figure or Table herein are referring to the Supplementary Figures and Tables disclosed in U.S. Provisional Patent Application Ser. No. 62/447,417, filed Jan. 17, 2017, which is specifically incorporated by reference.

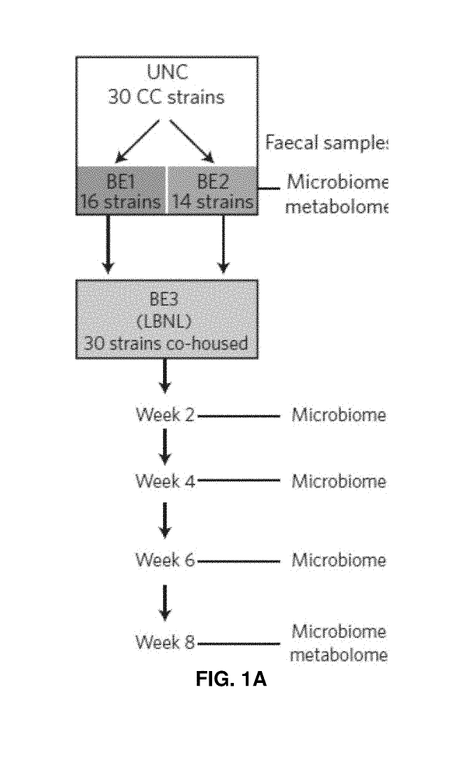

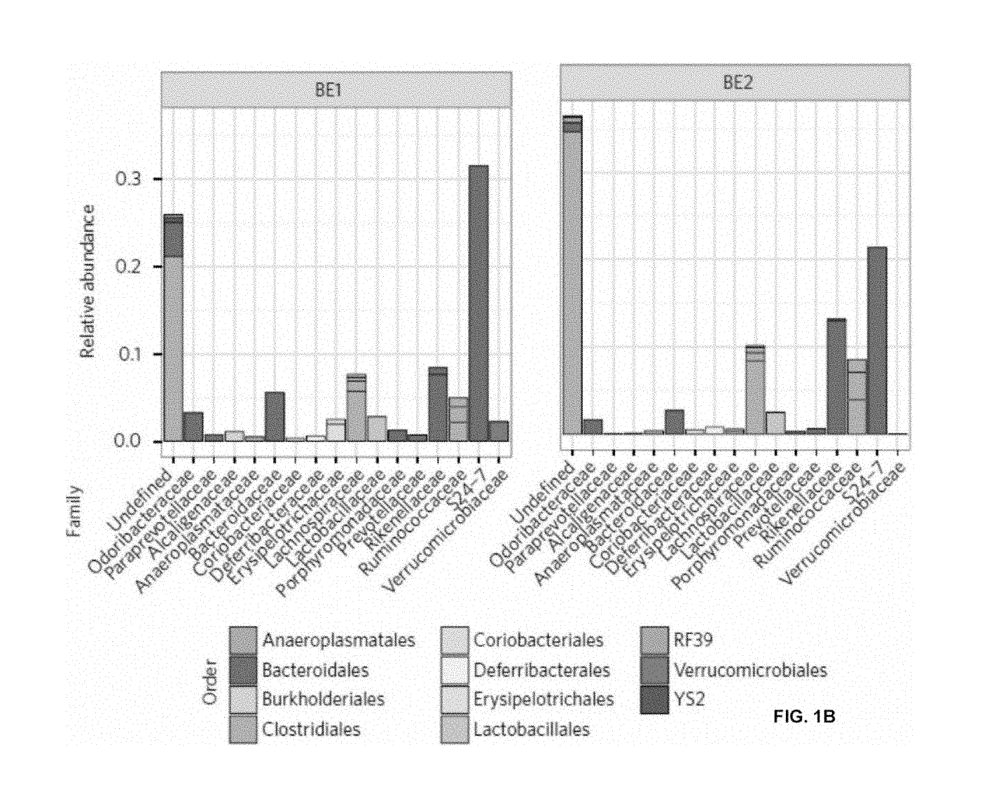

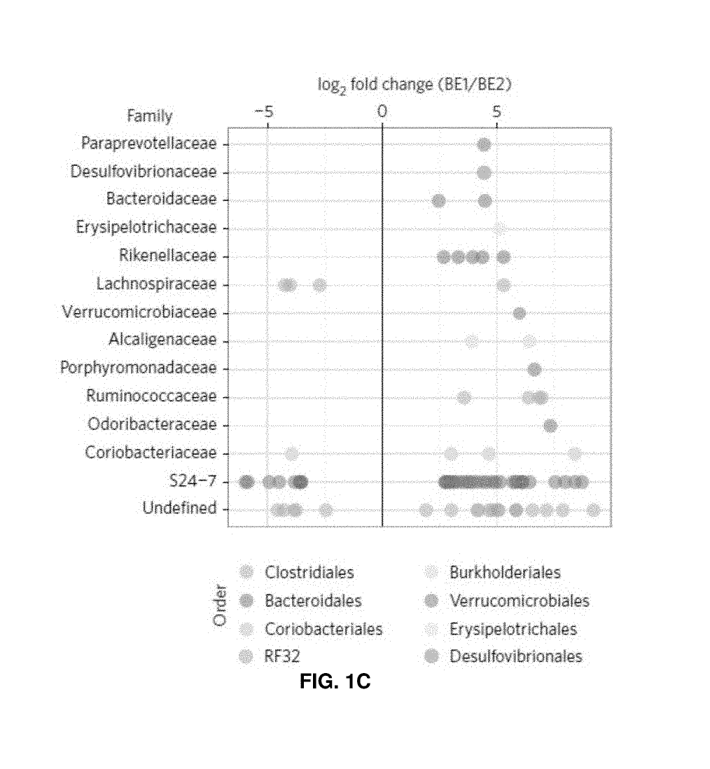

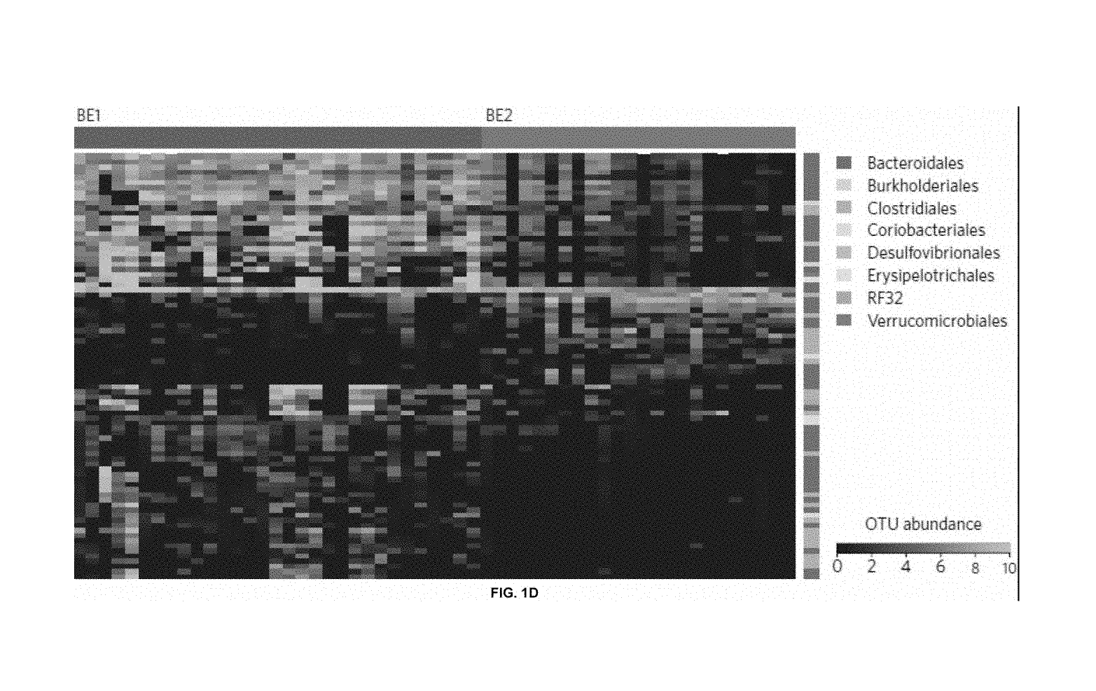

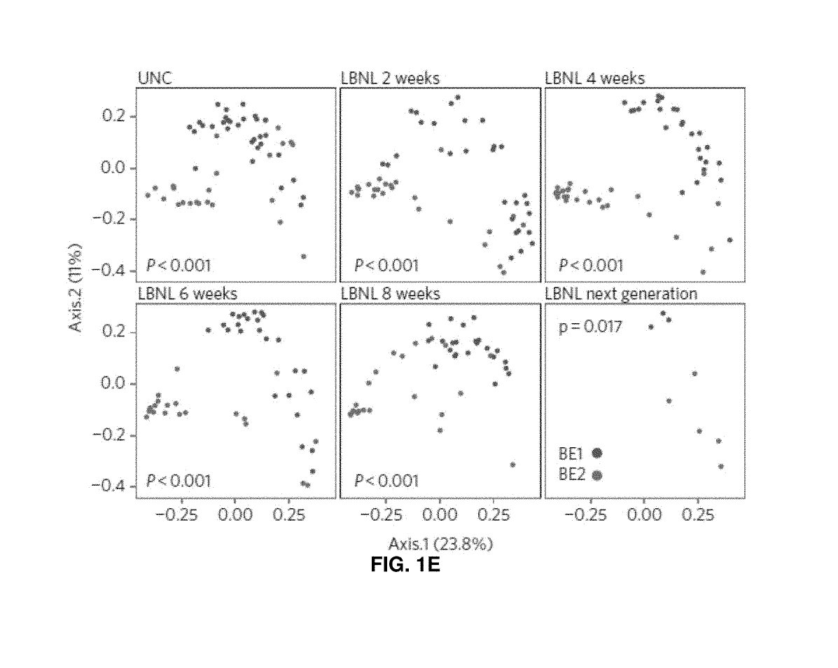

Supplementary FIG. 1. Effect of the built environment on the structure of the gut microbiome. a, Hierarchical clustering of mouse fecal microbial composition of the top 300 most abundant OTUs. Built environments (BE) are indicated in blue (BE2; barrier facility) and red (BE1; SPF facility). b, Stability of the gut microbial structure 2, 4, 6 and 8 weeks after transfer from BE1 and BE2 to BE3. Samples were separated by BE at birth (BE1: left; BE2: right). Relative abundances of the 20 most common families (representing >95% of the total data) over time are shown.

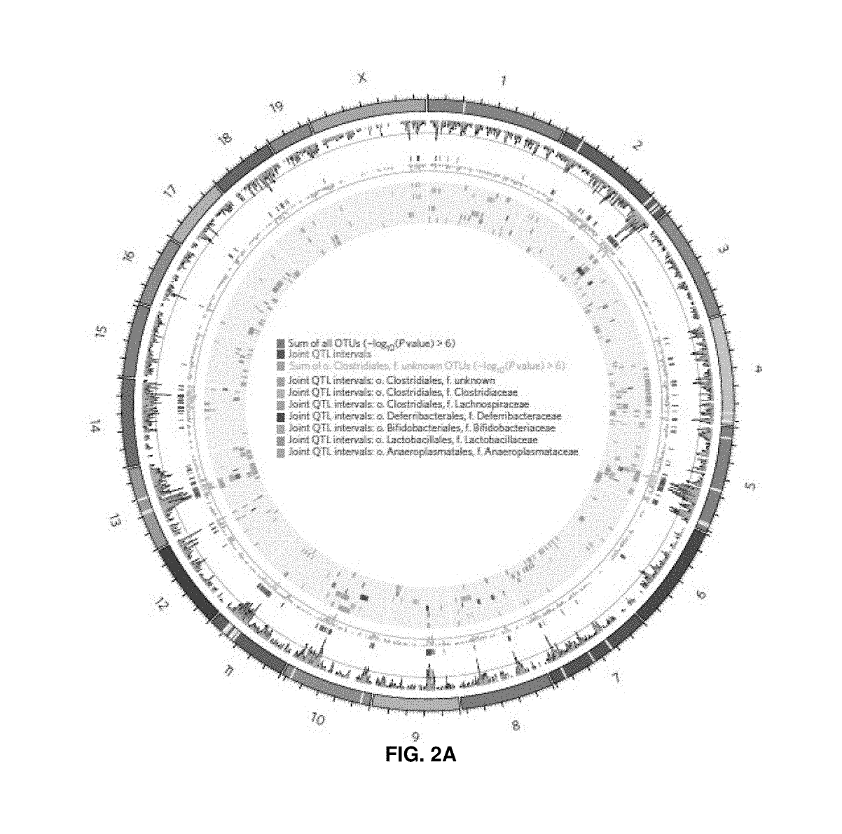

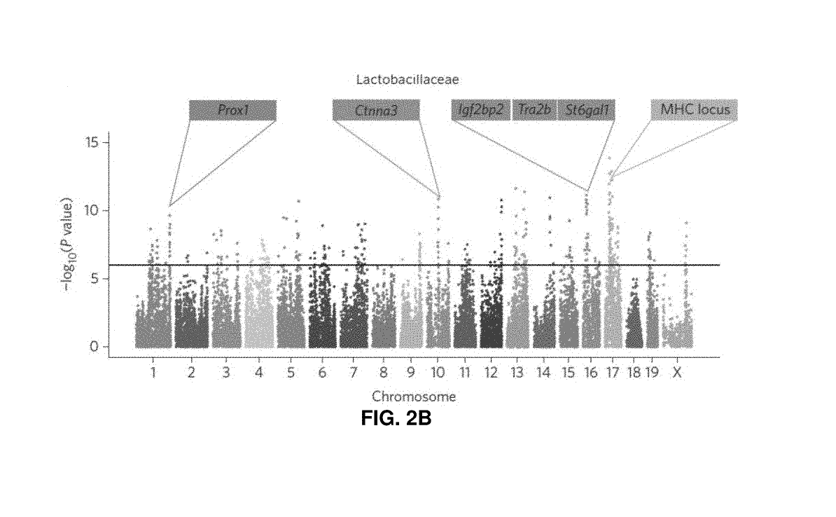

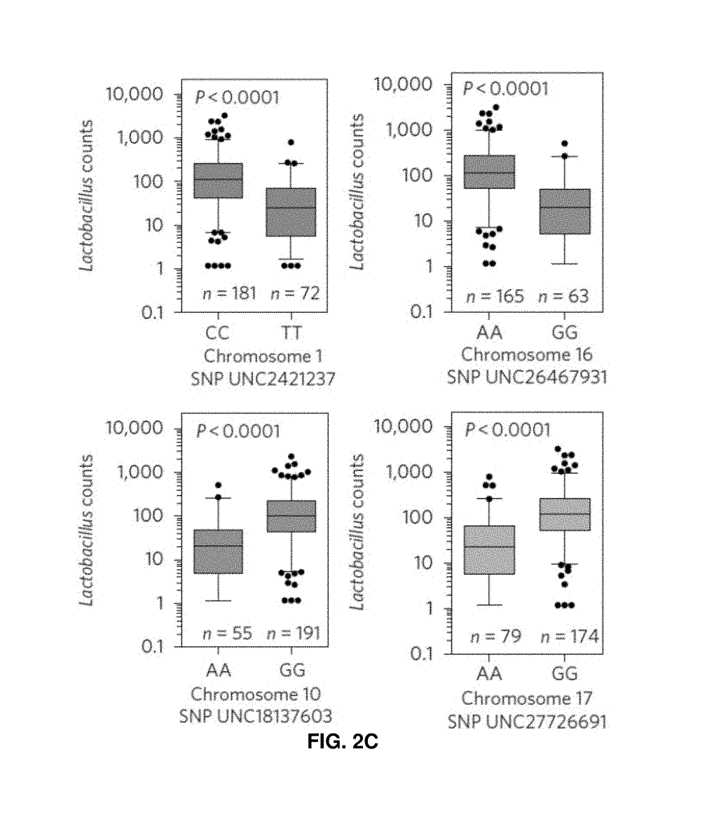

Supplementary FIG. 2. Polar dendrogram of OTU counts and metabolome profiles. Polar dendrograms using Bray-Curtis distance metric were generated for all mice based on normalized OUT counts (top) and metabolite abundance levels (bottom). Labels are colored by strain and include built environment at time of collection, the collection time-point at BE3 (2, 4, 6 or 8 weeks after arrival; microbiome only), the strain name (not including the original derivation location (Tau, Geni), but all from Unc) and sex.

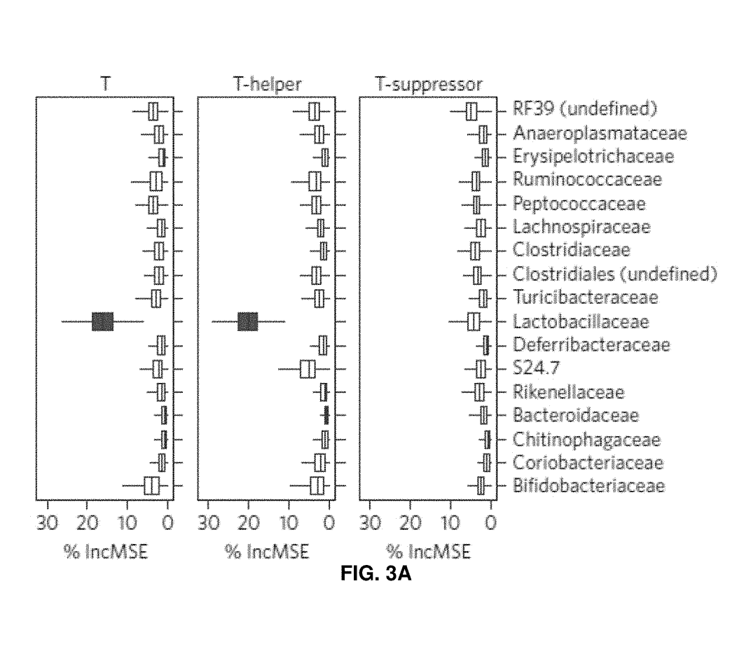

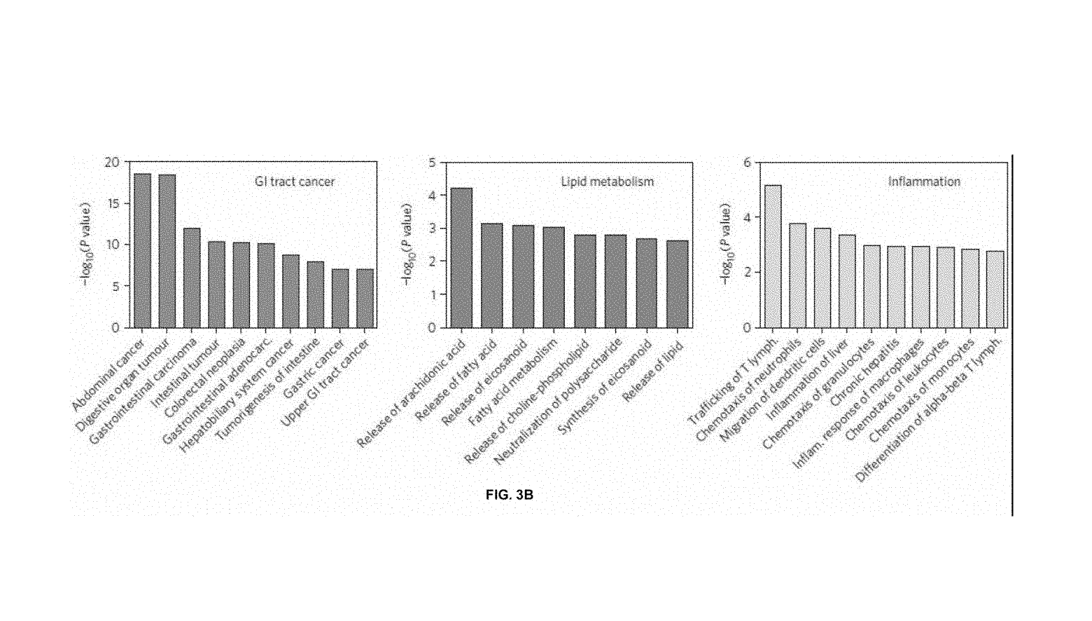

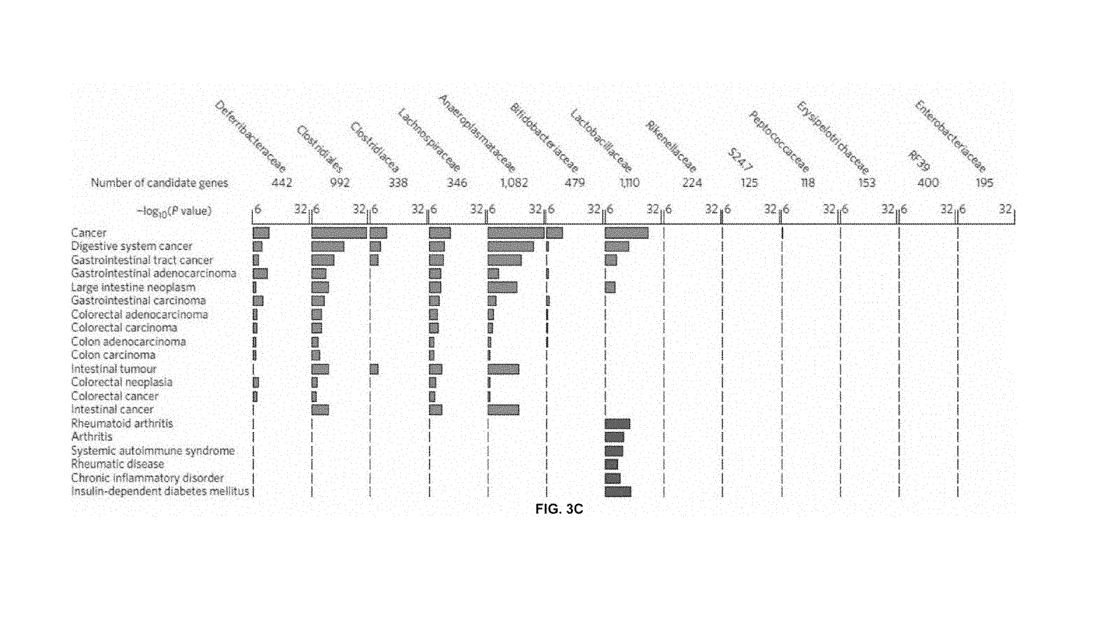

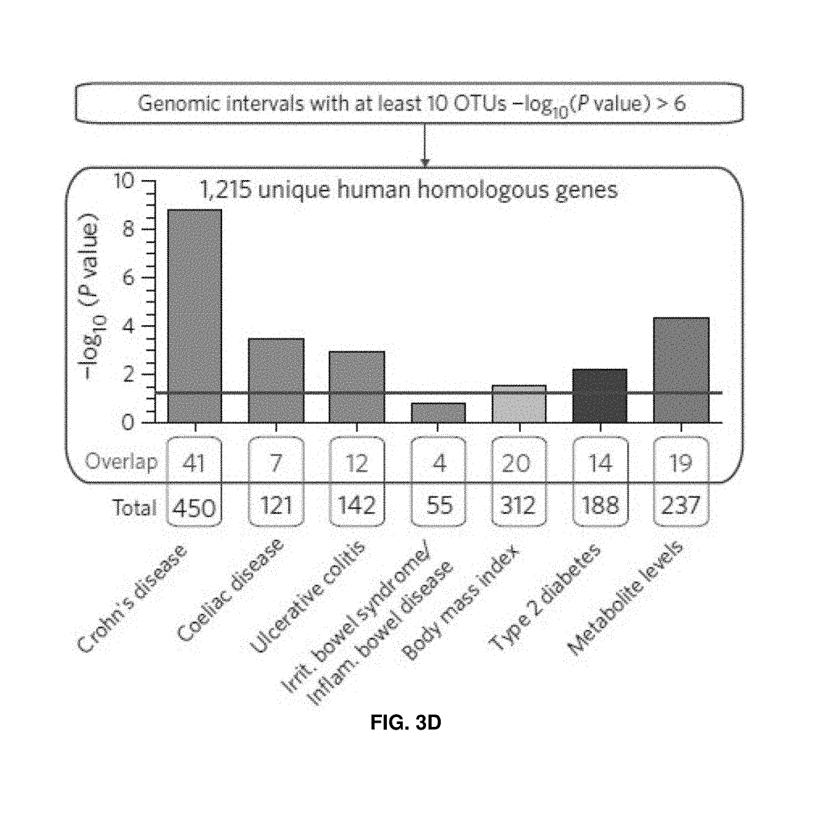

Supplementary FIG. 3. Visual representation of OTU correlation network. a, Network edges (gray lines) connect significantly correlated OTUs (ovals; colored at the family level). Correlation (>0.5) based on Spearman rank (p<1E-10). b, The genomic intervals with significant linkage (−log 10 (p-value)>6) to individual OTUs are shown within three individual correlation sub-networks.

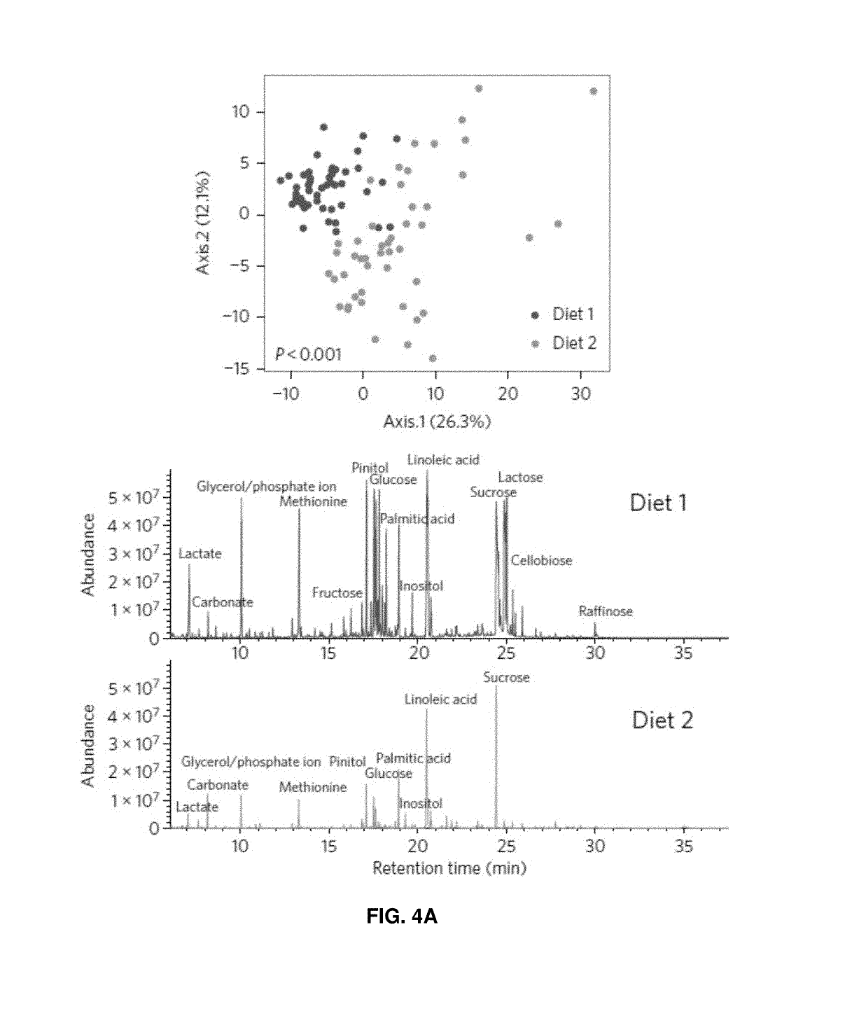

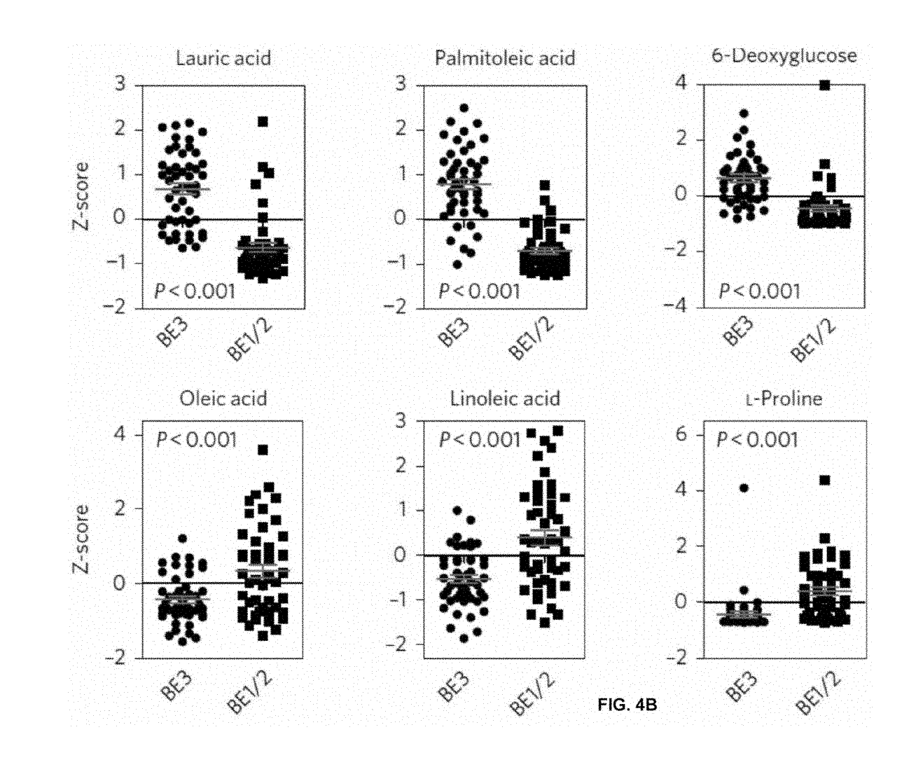

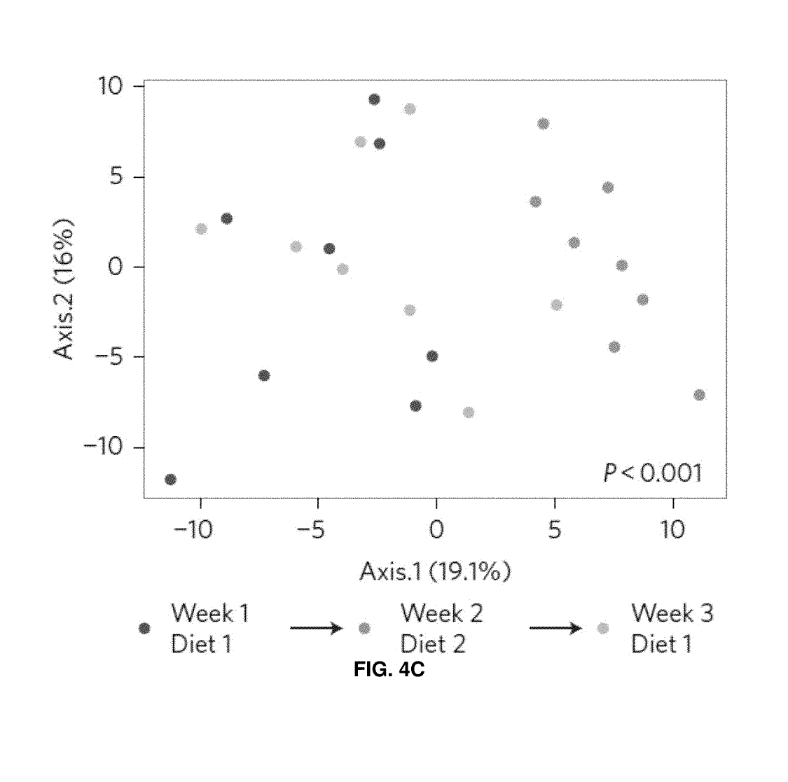

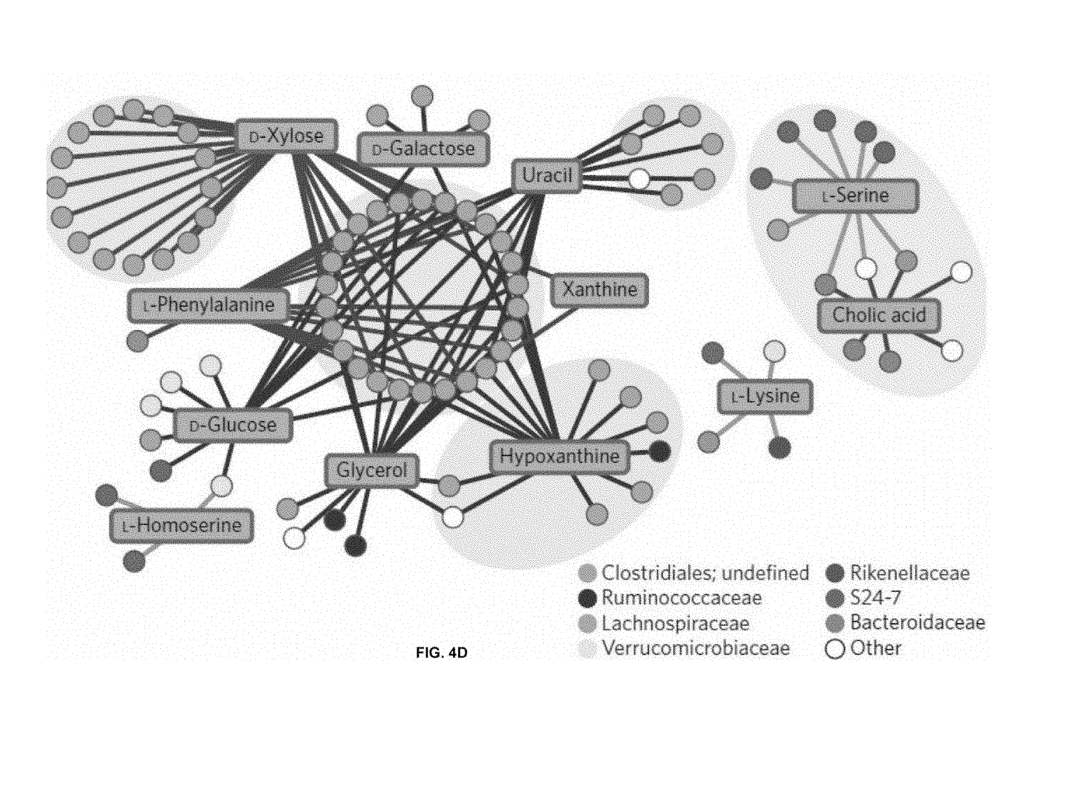

Supplementary FIG. 4. Association of microbial abundance with host phenotypes. a, Random forest analysis to assess the association between microbial abundance at the family level and mouse peripheral blood B cell counts, body weight and rotarod performance. Significant associations are indicated in green (p<0.05). However, these associations were not significant after adjusting for multiple comparisons using Benjamini-Hochberg b, Random permutation analysis to assess significance of association between microbial abundance and host phenotypes. Upper whiskers extend to the highest value that is within 1.5*inter-quartile range (IQR). The lower whiskers represent the lowest value within 1.5*IQR.

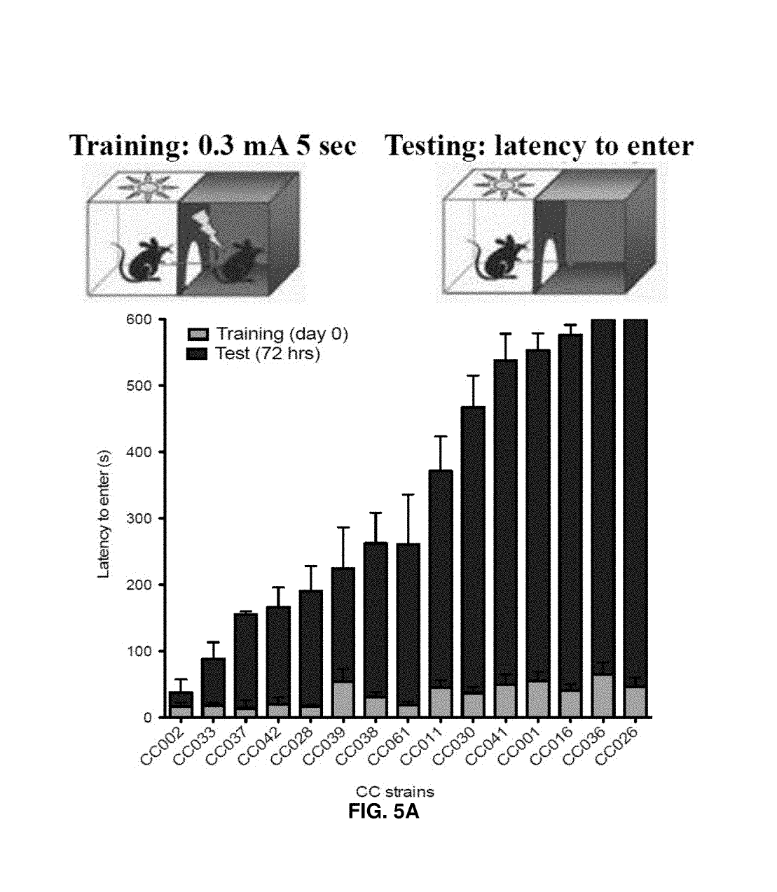

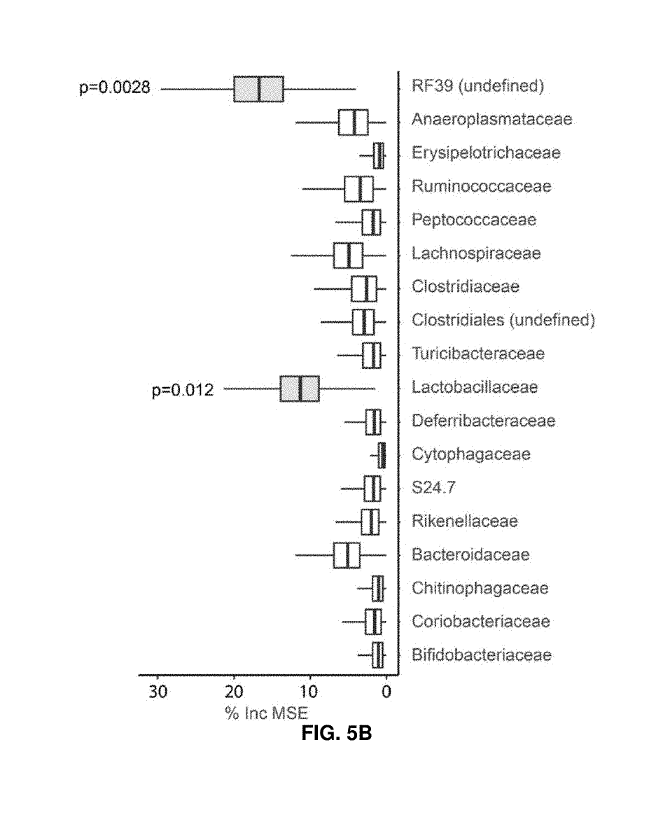

Supplementary FIG. 5. Metabolite profiles are controlled by the local diet. a, Hierarchical clustering of fecal metabolite levels in 24 CC strains maintained on two distinct diets (BE1 and 2; Labdiet Picolab 3500 and BE3; Labdiet Prolab 5053). b, Microbial abundance was measured in fecal samples of five CC strains (males and females were analyzed separately for each strain) maintained for one week on standard chow (Labdiet Picolab 5053; Diet 1), one week on autoclaved chow (Labdiet Prolab 3500; Diet 2) followed by one week on standard chow (p=0.273, R2=0.0079 between two diets, ADONIS).

Supplementary FIG. 6. Shifts in community metabolic capacity explain observed variation in non-dietary metabolites. a, Proportions of dietary and non-dietary metabolites whose measured variation is consistent (positively correlated) or contrasting (negatively correlated) with community metabolic potential (as predicted by metabolic modeling). b, Correspondence between variation in community metabolic potential and variation in measured metabolite concentration across all samples and across sample subsets from each diet and facility. The far right bar indicates whether each metabolite was detected in the chow from either facility.

Supplementary FIG. 7. Potential taxonomic contributors to metabolic modeling-based metabolite predictions. The three bars on the left indicate whether each metabolite's variation was consistent or contrasting with the predicted community metabolic potential across all samples and across sample subsets from each facility (BE1 and BE2: Diet 2; BE3: Diet 1) and diet. The right grid shows the number of OTUs in each taxonomic category identified as potential contributors to community metabolism, based on consistent variation patterns and metabolic capacity.

Supplementary FIG. 8. Potential taxonomic contributors to metabolite abundance for mice housed at BE3. Potential taxonomic contributors to metabolite variation for mice maintained on Labdiet Prolab 5053 (BE3). Individual OTUs shown (circles; colored at the family level) are those whose metabolic capacity and variation across samples are consistent with the entire community metabolic potential and with measured variation in the linked metabolites (squares). Green, orange and pink clouds behind OTU sub-networks indicate Clostridiales, Bacteroidales or Lactobacillales enrichment. Edge color indicates whether a given OTU potentially impacts a certain metabolite variation via synthesis (blue edges), degradation (green edges), or both (purple edges).

Supplementary FIG. 9. Relative contribution of built environment (BE) and genetics to microbial abundance. The percent deviance (% Dev) of microbial abundance across CC strains explained by BE and genetic factors are shown. Genetic factors contribute more than built environment (BE) to the total microbiome variation. Inset shows an example of the effect of a SNP on chromosome 5 on Lactobacillus abundance, where BE has no effect. Upper whiskers extend to the highest value that is within 1.5*inter-quartile range (IQR). The lower whiskers represent the lowest value within 1.5*IQR.

Supplementary FIG. 10. Summary plots from permutation of 16S data to obtain FDR estimates for QTL analyses. We performed 1,000 permutations of strain identifiers and then computed the Mann-Whitney U at each SNP for (A) data combined at the family level, (B) 15 OTUs in the lower and (C) upper quintiles of sum of p-values across all SNPs and (D) the 15 OTUs with the lowest overall sum of p-values.

Supplementary Table 1. Meta data of samples used in 16S and metabolite analysis.

Supplementary Table 2. Normalized amplicon abundance. See separate excel sheet found at website for: nature.com/articles/nmicrobio12016221#supplementary-information, incorporated by reference.

Supplementary Table 3. Differentially abundant fecal operational taxonomic units (OTUs) between animal facility built environments (BE1 vs BE2).

Supplementary Table 4. P-values for each genetic locus obtained using Mann-Whitney U test for all OTUs. See separate excel sheet found at website for: nature.com/articles/nmicrobio12016221#supplementary-information, incorporated by reference.

Supplementary Table 5. Joint QTL intervals and candidate genes. See .txt table attached.

Supplementary Table 6. Linkage analysis of microbial families. See separate excel sheet found at website for: nature.com/articles/nmicrobio12016221#supplementary-information, incorporated by reference.

Supplementary Table 7. Candidate genes in genetic loci associated with specific microbial families.

Supplementary Table 8. Human genome-wide association studies of microbiome associated diseases.

Supplementary Table 9. Original intensity of metabolomics data of the detected metabolites from BE1/2 and BE3 mouse chow.

Supplementary Table 10. Metabolomics data including original intensity of the detected metabolites from murine feces and their zscored transformed values in separate tabs. See separate excel sheet found at website for: nature.com/articles/nmicrobio12016221#supplementary-information, incorporated by reference.

Supplementary Table 11. Metabolite profiles in fecal samples of four CC strains maintained on different diets. See table in .txt format attached.

Supplementary Table 12. A list of all metabolites assayed and analyzed in terms of community metabolic potential for each subset of the data, detailing correlations between metabolomics data and community metabolic potential scores and potential taxonomic contributors. See separate excel sheet found at website for: nature.com/articles/nmicrobio12016221#supplementary-information,incorporated by reference.

Slides 1-13 showing the objectives and accomplishments of the study.

### EXAMPLE 1

Key objectives of the study: Determine baseline impacts of early exposure, diet and host genetics on murine behavior, gut microbiome and gut metabolome. Determine impact of stress (e.g. altered day/light cycles) on murine host-microbe interactions. Determine role of specific microbes or metabolites in the observed host response to stress. Develop and apply novel imaging tools for determination of spatial arrangement of key microbes and/or metabolites to gut epithelial surfaces.

To decipher the respective contributions of host genetics, early life history and diet on the gut microbiome we leveraged 30 independent, genetically distinct Collaborative Cross (CC) mouse strains (FIG. 1A and Supplementary Table 1), a large multi-parental panel of recombinant inbred strains with defined single nucleotide polymorphisms (SNPs) that captures ˜90% of the known variation in laboratory mice. Sixteen strains were maintained in a specific pathogen-free (SPF) facility (Built Environment 1, BE1), and 14 additional strains were maintained in a barrier facility that screens for additional infectious agents, including Pasteurella pneumotropica and Helicobacter (Built Environment 2, BE2). Mice were fed the same water and food sources at both locations. Faecal samples were collected at 12 weeks of age (FIG. 1A) and the gut microbiome composition was characterized by sequencing 16S rRNA genes (V4 hypervariable region; Supplementary Table 2).

Unsupervised hierarchical clustering of the 300 most abundant operational taxonomic units (OTUs) revealed two main clusters, each associated with a specific BE (Supplementary FIG. 1a), indicating a strong effect of BE on microbiome composition. We observed differences in the relative abundances of specific microbial families (FIGS. 1B to 1D and Supplementary Table 3). Specifically, there were higher relative abundances of Alcaligenaceae, Verrucomicrobiacae, Erysipelotrichaceae and Deferribacteracea and lower relative abundances of Clostridiales in BE1 compared to BE2. The consistency of BE-specific microbial signatures across genetically diverse CC strains strongly suggests that the BE influence on the gut microbe composition is at least in part independent of genetic background.

Mice from the same 30 CC strains were then transferred to a third SPF facility (BE3) to investigate the stability of the gut microbiome in response to a new environment where all mouse strains were subjected to the same conditions of husbandry. Faecal samples were collected from mice at 2, 4, 6 and 8 weeks after arrival at BE3, and the faecal microbiome was profiled by 16S sequencing (FIG. 1A). Principal coordinate analysis (PCoA) using Bray-Curtis distance revealed that the microbiome was stable and remained largely defined by the source BE, even after 8 weeks in BE3 (FIG. 1E and Supplementary FIG. 1b). To further assess the persistence of the source building effect on the microbiome, we performed 16S rRNA gene sequencing of faecal samples from eight CC strains born at BE3 (that is, second generation). PCoA confirmed that mice born at BE3 maintained their parents' source building microbial signature (FIG. 1E). We conclude that the gut microbiome, at least partially shaped by early life history, is persistent and shared between parents and their offspring, even when challenged with a new environment. Future studies need to be conducted to investigate the stability of the source building microbial signature across multiple generations.

One of our main aims was to determine the influence of host genetics on the gut microbiome. Hierarchical clustering of OTUs revealed that the majority of samples collected from the same strains of mice at different time points clustered together, suggesting that host genetics plays a role in determining the gut microbial composition (Supplementary FIG. 2). To identify genetic loci associated with specific OTUs, we performed independent quantitative trait loci (QTL) analyses by interrogating 50,107 SNPs across the genome (Supplementary Table 4). This analysis identified 169 joint QTL intervals that were significantly associated with the abundances of ten or more OTUs (−log₁₀(P value)>6) (FIG. 2A and Supplementary Table 5) and revealed a complex host genetic architecture of the gut microbiome composition (Supplementary FIG. 3). These genetic linkages were predominantly driven by the most abundant representative in the OTU data—a member of the Clostridiales (family unknown, FIG. 2A, green track). Abundances of other bacterial families were also controlled by multiple genetic loci (FIGS. 2A to 2C and Supplementary Table 6). Interestingly, the major histocompatibility complex (MHC) locus on chromosome 17 was significantly (P<0.0001) linked to the abundance of Lactobacillaceae (FIGS. 2B and 2C), consistent with an earlier report showing that MHC variation shapes microbial communities in the mouse gut, in particular the genus Lactobacillus³. Our findings also support earlier reports showing an association of the gut microbiota with host genetic variations^{4,5,6,7}. However, on leveraging the CC mice we identified over a hundred novel genetic loci that impact the gut microbiome.

To investigate the association of the gut microbiome with host phenotypes and behavior, we measured body weight, rotarod performance and immune cell abundance in the mice. Random forest analysis indicated that Lactobacillaceae abundance was predictive of T cell counts in peripheral blood (FIG. 3A; adjusted P=0.02), driven predominantly by T-helper cell levels (adjusted P=0.00087) but not T-suppressor cell levels (FIG. 3A). Modest associations were found for B-cell counts, body weight and rotarod performance (Supplementary FIG. 4). These results are consistent with reports that (1) Lactobacillus consumption is associated with an increase in CD4 counts in patients with HIV^{8,9}. (2) Lactobacilli can regulate behavior in mice¹⁰ and (3) Lactobacilli can serve as natural enhancers of cellular immune responses¹¹. Our results, using a non-targeted approach to assess the microbiome, extend these findings by demonstrating that only Lactobacilli have statistically significant associations with T cell counts, emphasizing the importance of Lactobacilli for health of the host.

The QTL that were specifically associated with Lactobacillaceae abundance in mice displayed significant enrichment for genes implicated in autoimmune disorders such as diabetes and arthritis (FIG. 3C). Examples of candidate genes in QTL linking Lactobacillus QTL with human phenotypes (FIG. 2B) include Prospero Homeobox 1 (Prox1) on chromosome 1 (associated with type 2 diabetes, obesity and fasting glucose levels), Catenin Alpha 3 (Ctnna3) on chromosome 10 (associated with serum pyroglutamine metabolite levels and arrhythmogenic right ventricular dysplasia, familial 13) and Insulin Like Growth Factor 2 MRNA Binding Protein 2 (Igf2bp2), Transformer 2 Beta Homolog (Tra2b) and ST6 Beta-Galactoside Alpha-2,6-Sialyltransferase 1 (St6gal1) on chromosome 16 (associated with type 2 diabetes and colon adenocarcinoma and colorectal cancer)^{12,13,14,15,16}. These results suggest an important role for host regulation of Lactobacillaceae abundance in health and disease and are concordant with recommendations for the use of probiotics containing Lactobacillus species as adjunctive therapies for the treatment of rheumatoid arthritis and diabetes.^{17,18}

Candidate genes located within the boundaries of the 169 identified QTL (2,699 genes; Supplementary Table 5) were analyzed to assess additional human relevance. We found that genes controlling the abundance of specific members of the microbiome were significantly enriched in human gastrointestinal cancer (1.02×10⁻⁷<P<3.17×10⁻¹⁹), inflammatory responses (1.75×10⁻³<P<7.15×10⁻⁶) and lipid metabolism (2.38×10⁻³<P<6.35×10⁻⁵) (FIG. 3B), providing further evidence for the involvement of both host genetics and the gut microbiome in health and disease. Genome-wide association (GWA) analysis was used to identify the genetic loci associated with abundance of microbial taxa at the family level. We found 13 of the taxa associated with QTL that contained >100 genes (Supplementary Table 7), of which 7 were significantly enriched for human genes implicated in gastrointestinal tract cancer (FIG. 3C). Comparing the mouse genes to a previously compiled list of human disease-related genes identified by GWA studies (GWAS)¹⁹, a significant overlap was observed for genes associated with Crohn's disease, coeliac disease, ulcerative colitis and type 2 diabetes (FIG. 3D and Supplementary Table 8). We conclude that candidate mouse genes within the loci identified as controlling microbiome abundance exhibit significant overlap with human genes previously linked to disease states, suggesting that the microbiome may contribute to their aetiology.

Investigation of the faecal metabolite composition allowed us to determine the influence of early life environment and diet on the gut metabolome. For these analyses we focused on 24 CC strains that were housed in BE1 and BE2 (fed Diet 2, Labdiet Prolab 3500) and in BE3 (fed Diet 1, Labdiet Picolab 5053). Although the two diets have similar macronutrient compositions, the metabolite profiles are quite distinct (FIG. 4A, lower panel; Supplementary Table 9). Extracts from the stool samples were analyzed by gas chromatography-mass spectrometry (GC-MS) and metabolites were identified by comparison to a reference library containing mass spectral and retention index information for over 850 metabolites²⁰. A total of 122 unique metabolites were identified, including amino acids, sterols, mono- and disaccharides, glycolytic and tricarboxylic acid cycle intermediates, short- and long-chain fatty acids, and products of microbial metabolism. An additional 110 peaks were detected but not identified (Supplementary Table 10).

The metabolites significantly clustered by diet, with differences in relative abundances of proteinogenic amino acids, mono- and disaccharides, sterols and fatty acids driving the separation (FIGS. 4A and 4B, and Supplementary FIG. 5a). To validate that the gut metabolome is primarily influenced by diet, four CC strains were maintained on Diet 1 for one week, then on Diet 2 for one week, before switching back to Diet 1 for an additional week. Fresh faecal samples were collected at the end of each week for metabolome profiling (Supplementary Table 11). Although only subtle changes were observed in microbial abundance (Supplementary FIG. 5b; P=0.273), there was a major and reversible shift in the metabolome profile that coincided with dietary changes (FIG. 4C), demonstrating that the metabolome profile is largely influenced by diet.

We used a metabolic modelling-based framework, MIMOSA²¹, to identify metabolites whose variation across samples is explained by variation in the metabolic potential of the microbiome, based on differences in species composition and estimated gene composition. By applying MIMOSA to the pooled set of metabolome samples from both diets, we found that variation in dietary metabolites (compounds detected in chow pellets by metabolomics) was poorly explained by microbial community composition (Supplementary Tables 9 and 12). However, the variation in a high proportion of non-dietary metabolites (47.6%; 10 out of 21 metabolites not detected in chow) was consistent with predicted community metabolic potential (CMP), suggesting a substantial role for microbial metabolism in metabolite synthesis and/or degradation (Supplementary FIG. 6a). Specifically, the observed variation in many gut metabolites was consistent with the predicted CMP, including hypoxanthine, 1-homoserine, 5-hydroxyindoleacetate and cholate (Supplementary FIGS. 6 and 7). More metabolites varied consistently with predicted CMP in samples from the nutritionally simpler Diet 2, suggesting that the microbiome may have a larger and more direct impact on the faecal metabolome in this context. The predicted CMP was driven by the metabolic potential of a diverse set of taxa, including OTUs from the phyla Firmicutes, Bacteroidetes and Actinobacteria (FIG. 4D and Supplementary FIGS. 7 and 8). Interestingly, the measured concentrations of several metabolites present in one or both diets were negatively correlated with predicted CMP (mostly on the basis of microbial degradation enzymes; Supplementary FIG. 6b), indicating that food containing these metabolites could drive the expansion of microbes that use them efficiently. These findings highlight the combined impacts of diet and microbiome composition on the gut metabolome and the complex interactions between them.

Our studies using the CC mouse cohort and an integrated, systematic analysis paradigm revealed how gut microbiome composition and function are shaped by interactions between host genotype, early life environment and diet, and identified several host genetic loci that regulate microbial abundance. Using multivariate analysis we quantified the relative influence of environment and genetics on microbial abundance and determined that genetics plays a larger role than environment (Supplementary FIG. 9). This study provides a foundation for future investigations of how reciprocal interactions between host genotype, environmental factors, gut microbiome and metabolome compositions contribute to a wide spectrum of mammalian traits and disease susceptibility.

Methods

Mouse husbandry and faecal sample collection. Mice were obtained from the Systems Genetics Core Facility at the University of North Carolina (UNC)²². Before their relocation to UNC, CC lines were generated and bred at Tel Aviv University in Israel²³, Geniad in Australia²⁴ and Oak Ridge National Laboratory in the USA²⁵. All studies were performed on young adult mice (age 9-15 weeks). For each of 30 strains (for strain information and number of replicate samples see Supplementary Table 1), two males and two females were housed separately and maintained on PicoLab Rodent Diet 20 (5053). The number of CC strains used is sufficient to detect genetic association. The investigators were not blinded in the analysis of the phenotypes because the correct genotype of CC mice was needed to perform genotype-phenotype and phenotype-phenotype association analysis. Mice from different strains were always housed in different cages. We observed a subtle change in microbial composition in samples collected 16 h after a cage change compared to <2 h. However, to collect sufficient mouse faecal material for combined microbiome and metabolomic analysis, all faecal samples were consistently collected from each cage, avoiding areas clearly contaminated with urine, 16 h after cage change at 2, 4, 6 and 8 weeks after arrival at Lawrence Berkeley National Laboratory (LBNL). All animal procedures were approved by the UNC Chapel Hill or LBNL Institutional Animal Care and Use Committees.

Faecal samples were stored at −80° C. for downstream metabolite and microbial analyses. Faecal samples from different strains were collected in the same way to avoid collection and storage biases. Genotyping data for CC mice were obtained from UNC (website for: csbio.unc.edu/CCstatus/index.py).

Faecal samples were collected from a different cohort of genetically identical young adult mice at UNC Chapel Hill (maintained on Labdiet Prolab 3500) to determine the effect of environment on the faecal microbiome and metabolome. Faecal samples were then manually homogenized on ice with a micropestle, 0.25 g was used for DNA isolation, 0.05 g for metabolite extraction and the remainder stored at −80° C.

Microbiome analyses. Genomic DNA was extracted from 0.25 g of the homogenized faecal samples using the PowerSoil DNA Isolation Kit (website for: mobio.com/) according to the manufacturer's instructions. PCR amplification of the V4 region of the 16S rRNA gene was performed using the protocol developed by the Earth Microbiome Project (website for: press.igsb.anl.gov/earthmicrobiome/empstandard-protocols/16s/) and described in ref. 26 using updated primers described in ref. 27. Amplicons were sequenced on an Illumina MiSeq using the 150 base pair (bp) MiSeq Reagent Kit v2 (website for: illumina.com/) according to the manufacturer's instructions.

QIIME 1.9.1 was used to join, quality filter and demultiplex libraries from three MiSeq runs^{28,29}. VSEARCH 1.1.3 was used to dereplicate, sort by abundance, remove single reads and then to cluster at 97% similarity. VSEARCH was also used to check these clusters for chimaeras and construct an abundance table by mapping labelled reads to chimaera-checked clusters^{30,31,32}. Taxonomy was assigned to the centroid of each cluster using the Qiime script assign_taxonomy.py and the Greengenes database. The centroids were aligned to Greengenes with PyNast and a phylogenetic tree was constructed using FastTree^{33,34,35}.

QTL mapping. 16S data from 30 strains (253 samples) were used in the analysis. OTUs showing significant differences in abundance (t-test P<0.01 or DESeq2 adjusted P<0.01) based on source building were filtered from the data, leaving 644 OTUs (of a total of 3,786). These OTUs represented 15% of total sequencing data. Genetic association was assessed for each OTU separately, and OTUs were merged at the family level. Genotype data for 77,597 SNPs were obtained from the UNC Systems Genetics Core website (website for: csbio.unc.edu/CCstatus/index.py) and filtered for minor allele frequency >4 of the 30 CC strains, leaving 50,107 SNPs. At each SNP, normalized OTU counts from CC samples were assigned to their respective alleles. We then used the Mann-Whitney U test⁴⁰ to test the significance of associations between OTU abundance and allele classes at each SNP. We used permutation to ascertain the significance of our results on an individual OTU basis to obtain a nonparametric estimate of the false discovery rate (FDR), as follows. For data combined at the family level, 15 OTUs in the upper and lower quintiles of P value sums across all SNPs (a proxy for signal of genetic association) and 15 OTUs with the lowest sums of P values, we performed 1,000 permutations of strain identifiers and then computed the same statistic at each SNP (Supplementary FIG. 10). This confirmed that a cutoff of −log₁₀(P value)>6 was a conservative threshold with a genome-wide FDR of <1%.

QTL were defined by merging SNPs with −log₁₀(P value)>6 within 1 Mb into multi-SNP intervals. Those with only one SNP were removed, and the remaining QTL boundaries were extended to the adjacent neighboring SNPs. Putative candidate genes were defined as those genes (gencode.vM7⁴¹) partially overlapping with or contained within a QTL locus. The list of candidate genes was analyzed using Ingenuity Pathway Analysis, converted into human homologues using the MGI homology resources⁴² (downloaded October 2015) and compared to human GWAS downloaded from website for: ebi.ac.uk/gwas (ref. 19). The significance of overlap between mouse and human candidate genes was calculated using ConceptGen (website for: conceptgen.ncibi.org/core/conceptGen/index.jsp). Visualization of genetic association and QTL was performed in R using ggplot2 and ggbio and with Circos^{39,43,44}.

OUT associated with host phenotypes. Whole blood was collected into ethylenediaminetetraacetic acid-coated tubes at 12 weeks of age in a cohort of 267 mice across 16 CC strains. Complete blood cell counts were acquired using a HemaVet950FS. Lymphocyte subpopulations were identified by fluorescence-activated cell sorting (FACS) using cell-specific markers for B cells, T cells, T-helper and T-suppressor cells. Antibodies (BD Biosciences) used for this analysis were rat anti-mouse CD3-PE, rat anti-mouse CC45R/B220 PerCP, rat anti-mouse CD8a antibody APC and rat anti-mouse CD4 antibody Alexa 488. The percentages of cells in blood were determined on a BD FACS Calibur (Becton Dickinson) and data were analyzed with FlowJo software (Tree Star). Body weight and rotarod performance were measured as described previously⁴⁵ at 10 weeks of age for a cohort of 365 mice across 16 CC strains.

We modelled data collected in mouse strains as statistically exchangeable to enable analysis in cases where we collected phenotypic and normalized 16S data on mice from the same strain, but not the same mice. Random pairs of phenotypic and normalized 16S data (combined at the family level) were sampled 1,000 times and each subjected to random forest regression analysis (microbial abundances as predictors, phenotype as the response vector). Analysis was performed using the R randomForest implementation⁴⁶ (ntree=1,000, all other parameters set to default). It is necessary to resample 1,000 times to model variance associated with random pairing under the exchangeability model. We then generated null distributions (n=1,000) by permutation of strain identifiers (after sampling to reproduce paired data). For each taxonomic family, observed and null importance measures (% IncMSE: % increase in mean squared error) were compared to determine significance. P values were computed as the natural nonparametric estimate of the likelihood of the observed distribution under the permuted distribution. Specifically, for each observed score uᵢ ∈ Q and the null distribution Q, we computed the rank of uᵢ ∈ Q, denoted r_{Q,i} and the empirical quantile under the null pᵢ=(1/n)r_{Q, i} where n=1,001, then our final P value is given by P-value=Σni=1(1/n)pi. Note that Pᵢ=(1/n)r_{Q,i}. This P value has the desirable property that it is bounded below by the sample size simulated for the null (and of course bounded above by 1). Given that it is nonparametric, it is conservative. Tukey boxplots of % IncMSE were generated using the default method in ggplot2 (ref. 39).

To estimate the proportion of OTU variation explained by genetics and source BE, SNPs were selected where sufficient statistical power exists for modelling (with comparable allelic frequencies for source BE1 and BE2 (0.4≤fraction of allele frequency in BE1 and BE2≤0.6) from joint QTL intervals). SNPs with ambiguous or heterozygous genotypes for any strain were filtered. For each interval, a representative SNP with the lowest sum of Mann-Whitney U P values (across OTUs) was selected. OTU counts were then modelled as a linear function of SNP genotype (105 SNPs) and source BE using the glm( )function in R. Data were subsampled (leaving out 20% of the data) and the model was fitted 100 times. For each OTU, mean percent deviance explained by BE and combined SNPs was reported.

Extraction of metabolites from faecal homogenates. Metabolites were extracted from mouse faecal samples using a methanol/sonication method (for strain information and number of replicate samples see Supplementary Table 1)⁴⁷. Briefly, portions of the homogenized samples were weighed and extracted with cold (−20° C.) methanol proportionally (1 ml solvent added per 100 mg homogenate) in a microcentrifuge tube. The average weight of the homogenized faecal samples was 69.3±26.3 mg (mean±standard deviation, s.d.) and the methanol extracts contained the same theoretical concentration of metabolites. A 100 μl volume of each methanol extract was transferred to glass vials and dried in a speed-vac concentrator (Labconco CentriVap Benchtop Vacuum Concentrator). Dried metabolite extracts were chemically derivatized using a modified version of the protocol used to create FiehnLib²⁰. Briefly, dried metabolite extracts were dried again to remove any residual water if they had been stored at −80° C. To protect carbonyl groups and reduce the number of tautomeric isomers, 20 μl of methoxyamine in pyridine (30 mg ml⁻¹) was added to each sample, followed by vortexing for 30 s and incubation at 37° C. with generous shaking (1,000 r.p.m.) for 90 min. At this point, the sample vials were inverted once to capture any condensation of solvent at the cap surface, followed by a brief centrifugation at 1,000 g for 1 min. To derivatize hydroxyl and amine groups to trimethylsilylated (TMS) forms, 80 μl of N-methyl-N-(trimethylsilyl)trifluoroacetamide (MSTFA) with 1% trimethylchlorosilane (TMCS) were then added to each vial, followed by vortexing for 10 s and incubation at 37° C. with shaking (1,000 r.p.m.) for 30 min. Again, the sample vials were inverted once, followed by centrifugation at 1,000 g for 5 min. The samples were allowed to cool to room temperature and analyzed the same day.

An Agilent GC 7890A coupled with a single quadrupole MSD 5975C (Agilent Technologies) was used and the samples were blocked and analyzed in random order for each experiment. An HP-5MS column (30 m×0.25 mm×0.25 μm; Agilent Technologies) was used for untargeted metabolomics analyses. The sample injection mode was splitless and 1 μl of each sample was injected. The injection port temperature was held at 250° C. throughout the analysis. The GC oven was held at 60° C. for 1 min after injection and the temperature was then increased to 325° C. by 10° C. min⁻¹, followed by a 5 min hold at 325° C. (ref. 48). The helium gas flow rates for each experiment were determined by the Agilent Retention Time Locking function based on analysis of deuterated myristic acid and were in the range of 0.45-0.5 ml min⁻¹. Data were collected over the mass range 50-550 m/z. A mixture of fatty acid methyl esters (FAMEs) (C8-C28) was analyzed once per day together with the samples for retention index alignment purposes during subsequent data analysis.

GC-MS raw data files were processed using the Metabolite Detector software, version 2.5 beta (ref. 49). Briefly, Agilent .D files were converted to netCDF format using Agilent Chemstation, followed by conversion to binary files using Metabolite Detector. Retention indices (RIs) of detected metabolites were calculated based on analysis of the FAMEs mixture, followed by their chromatographic alignment across all analyses after deconvolution. Metabolites were initially identified by matching experimental spectra to an augmented version of FiehnLib²⁰ (that is, the Agilent Fiehn Metabolomics Retention Time Locked (RTL) Library, containing spectra and validated retention indices for over 700 metabolites), using a Metabolite Detector match probability threshold of 0.6 (combined retention index and spectral probability). All metabolite identifications were manually validated to reduce deconvolution errors during automated data-processing and to eliminate false identifications. We propose that this approach results in a metabolite identification confidence of Level 1.5 (Level 1 is highest, Level 4 is lowest), according to the guidelines recommended by the Metabolomics Standards Initiative Chemical Analysis Working Group of the Metabolomics Society⁵⁰. The library used to identify metabolites was generated by an external laboratory, but this library contains both retention indices and mass spectra from analyses of authentic chemical standards and our analyses were performed using methods identical to those used to create the library. The NIST 14 GC-MS library was also used to cross-validate the spectral matching scores obtained using the Agilent library and to provide identifications of unmatched metabolites (Level 2 identifications). The three most abundant fragment ions in the spectra of each identified metabolite were automatically determined by Metabolite Detector and their summed abundances were integrated across the GC elution profile; fragment ions due to trimethylsilylation (that is, m/z 73 and 147) were excluded from the determination of metabolite abundance. A matrix of identified metabolites, unidentified metabolite features (characterized by mass spectra and retention indices and assigned as ‘unknown’; Level 4 identifications) and their abundances was created for subsequent data analysis. Features resulting from GC column bleeding were removed from the data matrices before further data processing and analysis.

Metabolic modeling-based taxonomic and metabolomic integration. We produced a closed-reference OTU table using VSEARCH to align reads from all 77 samples with both sequencing and metabolomics data to the preclustered Greengenes database. We rarefied the OTU table to 4,000 reads and used it as input to MIMOSA (website for: elbo.gs.washington.edu/software_MIMOSA.html), a framework for integrating taxonomic and metabolomic microbiome data²¹. MIMOSA uses genomic data, metabolic information and taxonomic composition to predict the community-wide biosynthetic and degradation potential for each metabolite in each sample and identifies metabolites whose variation across samples is consistent with (and can be explained by) variation in this predicted metabolic potential. Metagenome content was inferred for each sample using PICRUSt⁵¹ and normalized using MUSiCC⁵². From these data, a community-wide metabolic model was constructed for each sample and community metabolic potential (CMP) scores were calculated, representing the relative capacity of the predicted community enzyme content in that sample to synthesize or degrade each metabolite. We then compared variation in these scores across samples to variation in measured metabolite concentrations using a rank-based Mantel test, to identify metabolites for which variation in concentration across samples is positively correlated (consistent) with variation in community metabolism (as predicted by the CMP scores), using a local FDR q-value less than 0.01 as the significance threshold. We similarly identified metabolites for which variation in concentration across samples is negatively correlated (contrasting) with CMP scores, with the same significance threshold. To identify potential contributing OTUs for each metabolite, we calculated the Pearson correlation between the CMP scores obtained for a given metabolite across samples using the entire community and the CMP scores generated based on each species by itself (that is, recalculating the metagenome content and CMP scores based solely on the abundance of this species). OTUs for which this correlation coefficient for a given metabolite was greater than 0.5 were classified as potential contributing OTUs for that metabolite. Additional details about this computational framework have been described previously²¹.

Data availability. Sequence data are available at the Qiita management platform (website for: qiita.ucsd.edu/study/description/10500). Scripts to replicate this analysis are available at website for: github.com/pnnl/jansson_snijders_collaborative_cross. All raw GC-MS data are available via the MetaboLights metabolomics data repository (website for: ebi.ac.uk/metabolights/MTBLS345, ID MTBLS345). The data and sequences provided at these repositories are hereby incorporated by reference.

References Cited:
1. Clemente, J. C., Ursell, L. K., Parfrey, L. W. & Knight, R. The impact of the gutmicrobiota on human health: an integrative view. Cell 148, 1258-1270 (2012).
2. Collaborative Cross Consortium. The genome architecture of the CollaborativeCross mouse genetic reference population. Genetics 190, 389-401 (2012).
3. Kubinak, J. L. et al. MHC variation sculpts individualized microbial communities that control susceptibility to enteric infection. Nat. Commun. 6, 8642 (2015).
4. Goodrich, J. K. et al. Human genetics shape the gut microbiome. Cell 159, 789-799 (2014).
5. McKnite, A. M. et al. Murine gut microbiota is defined by host genetics and modulates variation of metabolic traits. PLoS ONE 7, e39191 (2012).
6. Benson, A. K. et al. Individuality in gut microbiota composition is a complex polygenic trait shaped by multiple environmental and host genetic factors. Proc. Natl Acad. Sci. USA 107, 18933-18938 (2010).
7. Benson, A. K. Host genetic architecture and the landscape of microbiome composition: humans weigh in. Genome Biol. 16, 203 (2015).
8. Anukam, K. C., Osazuwa, E. O., Osadolor, H. B., Bruce, A.W. & Reid, G. Yogurt containing probiotic Lactobacillus rhamnosus GR-1 and L. reuteri RC-14 helps resolve moderate diarrhea and increases CD4 count in HIV/AIDS patients. J. Clin. Gastroenterol. 42, 239-243 (2008).
9. Trois, L., Cardoso, E. M. & Miura, E. Use of probiotics in HIV-infected children: a randomized double-blind controlled study. J. Trop. Pediatr. 54, 19-24 (2008).
10. Bravo, J. A. et al. Ingestion of Lactobacillus strain regulates emotional behavior and central GABA receptor expression in a mouse via the vagus nerve. Proc. Natl Acad. Sci. USA 108, 16050-16055 (2011).
11. Mohamadzadeh, M. et al. Lactobacilli activate human dendritic cells that skew T cells toward T helper 1 polarization. Proc. Natl Acad. Sci. USA 102, 2880-2885 (2005).
12. Replication, D. I. G. et al. Genome-wide trans-ancestry meta-analysis provides insight into the genetic architecture of type 2 diabetes susceptibility. Nat. Genet. 46, 234-244 (2014).
13. Gong, Y. et al. PROX1 gene variant is associated with fasting glucose change after antihypertensive treatment. Pharmacotherapy 34, 123-130 (2014).
14. Yu, B. et al. Genome-wide association study of a heart failure related metabolomic profile among African Americans in the Atherosclerosis Risk in Communities (ARIC) study. Genet. Epidemiol. 37, 840-845 (2013).
15. Kim, H. J. et al. Combined linkage and association analyses identify a novel locus for obesity near PROX1 in Asians. Obesity 21, 2405-2412 (2013).
16. Manning, A. K. et al. A genome-wide approach accounting for body mass index identifies genetic variants influencing fasting glycemic traits and insulin resistance. Nat. Genet. 44, 659-669 (2012).
17. Alipour, B. et al. Effects of Lactobacillus casei supplementation on disease activity and inflammatory cytokines in rheumatoid arthritis patients: a randomized double-blind clinical trial. Int. J. Rheum. Dis. 17, 519-527 (2014).
18. Bordalo Tonucci, L. et al. Clinical application of probiotics in diabetes mellitus: therapeutics and new perspectives. Crit. Rev. Food Sci. Nutr. website for: dx.doi.org/10. 1080/10408398.2014.934438 (2015).
19. Hindorff, L. et al. A Catalog of Published Genome-Wide Association Studies; website for: ebi.ac.uk/gwas
20. Kind, T. et al. Fiehnlib: mass spectral and retention index libraries for metabolomics based on quadrupole and time-of-flight gas chromatography/mass spectrometry. Anal. Chem. 81, 10038-10048 (2009).
21. Noecker, C. et al. Metabolic model-based integration of microbiome taxonomic and metabolomic profiles elucidates mechanistic links between ecological and metabolic variation. mSystems 1, e00013-15 (2016).
22. Welsh, C. E. et al. Status and access to the Collaborative Cross population. Mamm. Genome. 23, 706-712 (2012).
23. Iraqi, F. A., Churchill, G. & Mott, R. The Collaborative Cross, developing a resource for mammalian systems genetics: a status report of the Wellcome Trust cohort. Mamm. Genome 19, 379-381 (2008).
24. Morahan, G., Balmer, L. & Monley, D. Establishment of ‘The Gene Mine’: a resource for rapid identification of complex trait genes. Mamm. Genome. 19, 390-393 (2008).
25. Chesler, E. J. et al. The Collaborative Cross at Oak Ridge National Laboratory: developing a powerful resource for systems genetics. Mamm. Genome. 19, 382-389 (2008).
26. Caporaso, J. G. et al. Ultra-high-throughput microbial community analysis on the Illumina HiSeq and MiSeq platforms. ISME J. 6, 1621-1624 (2012).
27. Walters, W. et al. Improved bacterial 16S rRNA gene (V4 and V4-5) and fungal internal transcribed spacer marker gene primers for microbial community surveys. mSystems 1, e00009-15 (2015).
28. Caporaso, J. G. et al. QIIME allows analysis of high-throughput community sequencing data. Nat. Methods 7, 335-336 (2010).
29. Aronesty, E. ea-utils: Command-Line Tools for Processing Biological Sequencing Data (Expression Analysis, 2011); website for: github.com/ExpressionAnalysis/ea-utils 30. Edgar, R. C. Search and clustering orders of magnitude faster than BLAST. Bioinformatics 26, 2460-2461 (2010).
31. Edgar, R. C., Haas, B. J., Clemente, J. C., Quince, C. & Knight, R. UCHIME improves sensitivity and speed of chimera detection. Bioinformatics 27, 2194-2200 (2011).
32. Rognes, T., Flouri, T. & Mahe, F. vsearch: VSEARCH Version 1.1.3 (2015); website for: zenodo.org/record/16153#.VwwcqxMrKuM
33. McDonald, D. et al. An improved Greengenes taxonomy with explicit ranks for ecological and evolutionary analyses of bacteria and archaea. ISME J. 6, 610-618 (2012).
34. Caporaso, J. G. et al. PyNAST: a flexible tool for aligning sequences to a template alignment. Bioinformatics 26, 266-267 (2010).
35. Price, M. N., Dehal, P. S. & Arkin, A. P. Fasttree 2—approximately maximumlikelihood trees for large alignments. PLoS ONE 5, e9490 (2010).
36. Love, M. I., Huber, W. & Anders, S. Moderated estimation of fold change and dispersion for RNA-seq data with DESeq2. Genome Biol. 15, 550 (2014).
37. Lozupone, C. & Knight, R. Unifrac: a new phylogenetic method for comparing microbial communities. Appl. Environ. Microbiol. 71, 8228-8235 (2005).
38. McMurdie, P. J. & Holmes, S. Phyloseq: an R package for reproducible interactive analysis and graphics of microbiome census data. PLoS ONE 8, e61217 (2013).
39. Wickham, H. ggplot2: Elegant Graphics for Data Analysis (Springer, 2010).
40. R-Core-Team. R: A Language and Environment for Statistical Computing (R Foundation for Statistical Computing, 2016); website for: R-project.org/.
41. Mudge, J. M. & Harrow, J. Creating reference gene annotation for the mouse C57BL6/J genome assembly. Mamm. Genome 26, 366-378 (2015).
42. Eppig, J. T. et al. The Mouse Genome Database (MGD): facilitating mouse as a model for human biology and disease. Nucleic Acids Res. 43, D726-D736 (2015).
43. Yin, T., Cook, D. & Lawrence, M. Ggbio: an R package for extending the grammar of graphics for genomic data. Genome Biol. 13, R77 (2012).
44. Krzywinski, M. et al. Circos: an information aesthetic for comparative genomics. Genome Res. 19, 1639-1645 (2009).
45. Mao, J. H. et al. Identification of genetic factors that modify motor performance and body weight using Collaborative Cross mice. Sci. Rep. 5, 16247 (2015).
46. Liaw, A. & Wiener, M. Classification and regression by randomForest. R News 2, 18-22 (2002).
47. Walker, A. et al. Importance of sulfur-containing metabolites in discriminating fecal extracts between normal and type-2 diabetic mice. J. Proteome Res. 13, 4220-4231 (2014).
48. Kim, Y. M. et al. Salmonella modulates metabolism during growth under conditions that induce expression of virulence genes. Mol. Biosyst. 9, 1522-1534 (2013).
49. Hiller, K. et al. Metabolitedetector: comprehensive analysis tool for targeted and nontargeted GC/MS based metabolome analysis. Anal. Chem. 81, 3429-3439 (2009).
50. Sumner, L. W. et al. Proposed minimum reporting standards for chemical analysis. Metabolomics 3, 211-221 (2007).
51. Langille, M. G. et al. Predictive functional profiling of microbial communities using 16S rRNA marker gene sequences. Nat. Biotechnol. 31, 814-821 (2013).
52. Manor, O. & Borenstein, E. MUSiCC: a marker genes based framework for metagenomic normalization and accurate profiling of gene abundances in the microbiome. Genome Biol. 16, 27 (2015).

### EXAMPLE 2

The association of the gut microbiome with memory is investigated in the Collaborative Cross mouse resource. Behavioral changes in mice is measured using the classical passive avoidance test, a fear motivated test, to assess memory. Mice are allowed to explore a white compartment. When animals cross over into the black compartment it receives a mild foot shock. Three days after the mild foot shock, mice are place in the white compartment again and the latency to enter the black compartment is measured. Mice with a long latency to enter the black compartment have “good memory”. Mice with a short latency to enter the black compartment have “bad memory”. Random Forest Analysis indicated that the abundance of: (1) k_Bacteria.p_Tenericutes.c_Mollicutes.o_RF39.f_(p=0.0028), and/or (2) k_Bacteria.p_Firmicutes.c_Bacilli.o_Lactobacillales.f_Lactobacillaceae (p=0.013), are predictive of “memory” using the passive avoidance test.

The above examples are provided to illustrate the invention but not to limit its scope. Other variants of the invention will be readily apparent to one of ordinary skill in the art and are encompassed by the appended claims. All publications, databases, references and patents cited herein are hereby incorporated by reference for all purposes.

What is claimed is:

1. A probiotic composition designed to enhance memory and/or reduce fear and/or pain of a host, the probiotic composition comprising viable or live cells of one or more of the family/order/strain selected from a group consisting of RF39, Lactobacillaceae, Bacteroidaceae, Lachnospiraceae, Anaeroplasmataceae, Ruminococcaceae, Clostridiales, Clostridiaceae, Rikenellaceae, Erysipelotrichaceae, Peptococcaceae, Turicibacteraceae, Deferribacteraceae, Cytophagaceae, Chitinophagaceae, Coriobacteriaceae, Bifidobacteriaceae, and S24.7.

2. The probiotic composition of claim 1 comprising viable or live cells of one or more of the family/order/strain RF39, Lactobacillaceae, Bacteroidaceae, Lachnospiraceae, Anaeroplasmataceae, Ruminococcaceae, Clostridiales, Clostridiaceae, or Rikenellaceae.

3. The probiotic composition of claim 2 comprising viable or live cells of strain RF39 and/or family Lactobacillaceae.

4. The probiotic composition of claim 1 comprising viable or live cells of the family/order/strain RF39, Lactobacillaceae, Bacteroidaceae, Lachnospiraceae, Anaeroplasmataceae, Ruminococcaceae, Clostridiales, Clostridiaceae, and Rikenellaceae.

5. A method of enhancing memory and/or reducing fear and/or pain of a host, the method comprising: administering a probiotic composition of claim 1 to a host, such that memory is enhanced, and/or fear and/or pain is reduced, for the host.

6. The method of claim 5, wherein the host is a mouse or a human.

7. The method of claim 5, wherein the administrating is an oral administration.
